(19) United States
(12) Patent Application Publication
(10) Pub. No.: US 2010/0204487 A1
(43) Pub. Date: Aug. 12, 2010

(54) PROCESS FOR MAKING THIOPHENE CARBOXAMIDE DERIVATIVES

(76) Inventor: Ian DAVIES, Princeton, NJ (US)

Correspondence Address: MERCK, P O BOX 2000, RAHWAY, NJ, 07065-0907, US

(21) Appl. No.: 12/669,880

(22) PCT Filed: Aug. 5, 2008
(86) PCT No.: PCT/US08/09389

§ 371 Date: Jan. 20, 2010

Related U.S. Application Data
(60) Provisional application No. 60/964,067, filed on Aug. 9, 2007.

(51) Int. Cl. C07D 333/38

(52) U.S. Cl. 549/72

(57) ABSTRACT

The invention encompasses a process for making a thiophene carboxamide derivative, which is an EP4 antagonist useful for treating pain and inflammation.

### BACKGROUND OF THE INVENTION

This invention relates to a process for making a thiophene carboxamide derivative, which is an EP4 antagonist useful for treating prostaglandin E mediated diseases, such as acute and chronic pain, osteoarthritis and rheumatoid arthritis. The compound is an antagonist of the pain and inflammatory effects of E-type prostaglandins and is structurally different from NSAIDs and opiates.

Three review articles describe the characterization and therapeutic relevance of the prostanoid receptors as well as the most commonly used selective agonists and antagonists: Eicosanoids: From Biotechnology to Therapeutic Applications, Folco, Samuelsson, Maclouf, and Velo eds, Plenum Press, New York, 1996, chap. 14, 137-154; Journal of Lipid Mediators and Cell Signalling, 1996, 14, 83-87; and Prostaglandins and Other Lipid Mediators, 2002, 69, 557-573.

Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, have effects on vascular homeostasis, reproduction, gastrointestinal functions and bone metabolism. These compounds may have a diminished ability to induce some of the mechanism-based side effects of NSAIDs which are indiscriminate cyclooxygenase inhibitors. In particular, the compounds are believed to have a reduced potential for gastrointestinal toxicity, a reduced potential for renal side effects, a reduced effect on bleeding times and a lessened ability to induce asthma attacks in aspirin-sensitive asthmatic subjects.

In The Journal of Clinical Investigation (2002, 110, 651-658), studies suggest that chronic inflammation induced by collagen antibody injection in mice is mediated primarily through the EP4 subtype of PGE₂ receptors. Patent application publications WO 96/06822 (Mar. 7, 1996), WO 96/11902 (Apr. 25, 1996) and EP 752421-A1 (Jan. 8, 1997) disclose compounds as being useful in the treatment of prostaglandin mediated diseases.

Thiophene carboxamide derivatives useful as EP4 antagonists and processes for making such compounds are disclosed in U.S. Provisional Application No. 60/837,252, filed on Aug. 11, 2006. Although the synthetic method disclosed in the above reference suffices to prepare small quantities of material, they suffer from a variety of safety issues, low yields or lengthy processes that are not amenable to large scale synthesis. The present invention describes an efficient and economical process for the preparation of thiophene carboxamide derivatives that is useful for the production of kilogram quantities of material for preclinical, clinical and commercial use.

### SUMMARY OF THE INVENTION

The invention encompasses a process for making a thiophene carboxamide derivative, which is an EP4 antagonist useful for treating pain and inflammation.

### DETAILED DESCRIPTION OF THE INVENTION

The invention encompasses a process for synthesizing a compound of Formula I

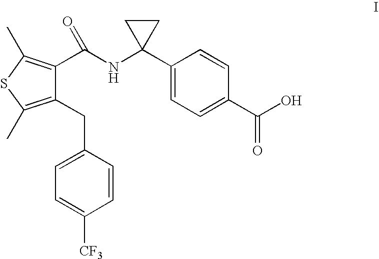

or a pharmaceutically acceptable salt thereof, comprising:
(a1) reacting a compound of Formula 5

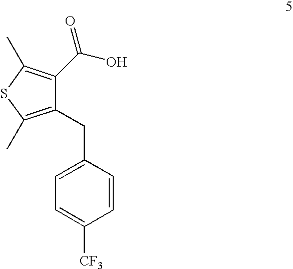

with a first chlorinating agent in the presence of dimethylformamide to yield the acid chloride of Formula 5a

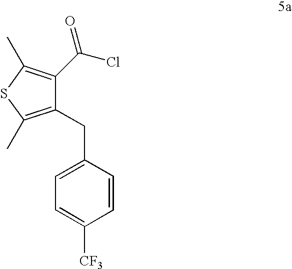

and reacting the compound of Formula 5a with a compound of Formula 7

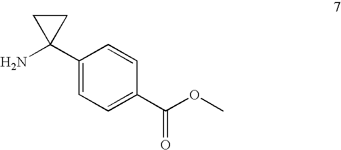

in the presence of an amine base to yield a compound of Formula 8

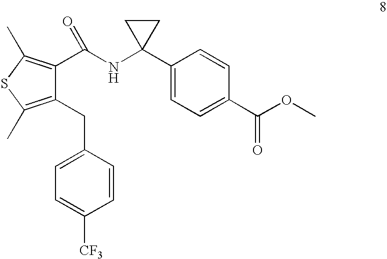

(b1) hydrolyzing the compound of Formula 8 with a strong base of formula X¹—OH or X²—(OH)₂, wherein X¹ is selected from the group consisting of: potassium, cesium, lithium, sodium and rubidium, and X² is selected from the group consisting of: barium, strontium and calcium, followed by acidification to yield the compound of Formula I; and
(e1) optionally reacting the compound of Formula I with a base to yield a pharmaceutically acceptable salt of the compound of Formula I.

For the above steps (a1) to (c1), the following amounts of the reagents may be used (relative to the first reagent in the process step): 1 to 2 equivalents of the first chlorinating agent, 0.01 to 0.1 equivalents of dimethylformamide, 0.8 to 1.5 equivalents of compound 7, 1 to 2 equivalents of the amine base, 1 to 10 equivalents of the strong base, 1 to 10 equivalents of the acid used in the acidification step, and 1 to 1.5 equivalents of the base used to form the pharmaceutically acceptable salt.

The term “first chlorinating agent” and “second chlorinating agent” independently mean a reagent that reacts with a carboxylic acid to form an acid chloride, such as thionyl chloride, phosphorous pentachloride and oxalyl chloride. An embodiment of the invention encompasses the process of the invention wherein the chlorinating agent is oxalyl chloride.

An amine base means for example primary, secondary, and tertiary amines, substituted amines including naturally occurring substituted amines, cyclic amines, for example, N,N-Diisopropylethylamine (Hünig's base), diethylamine, triethylamine and dipropylamine. An embodiment of the invention encompasses the process of the invention wherein the amine base is N,N-Diisopropylethylamine.

The term “acidification” means the addition of an appropriate acid, such as HCl.

The term “base” means an appropriate base which forms a pharmaceutically acceptable salt with the compound of Formula I. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. An embodiment of the invention encompasses the process of the invention wherein the base is diethylamine. Salts derived from inorganic bases include aluminum, ammonium, calcium, copper, ferric, ferrous, lithium, magnesium, manganic salts, manganese, potassium, sodium, zinc, and the like. Preferred salts derived from inorganic bases include sodium, potassium and calcium.

The invention also encompasses the process described in steps (a1) to (c1) above further comprising making the compound of Formula 5 by
(d1) reacting a compound of Formula 4

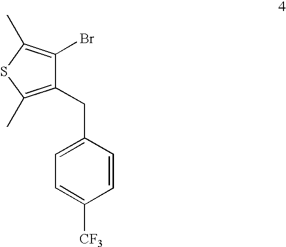

with an organolithium reagent in the presence of tetramethylethylenediamine in a solvent of methyl tertiary-butyl ether at a temperature of at or below about 55° C., and reacting the resulting mixture with CO₂ followed by an acid to yield a compound of Formula 5.

For the above step (d1), the following amounts of the reagents may be used (relative to the first reagent in the process step): 1 to 1.2 equivalents of the organolithium reagent, 1 to 1.5 equivalents of tetramethylethylenediamine, 5 to 20 L of methyl tertiary-butyl ether per kg of compound 4, 1 to 10 equivalents of CO₂ and 1 to 10 equivalents of the acid.

The term organolithium reagent means an organometallic compound with a direct bond between a carbon and a lithium atom. Examples include methyllithium, n-butyllithium and t-butyllithium. An embodiment of the invention encompasses the process of the invention wherein the organolithium reagent is n-butyllithium.

The term acid means any appropriate acid such as hydrochloric acid and sulfuric acid. In an embodiment of the invention, the acid is HCl.

The invention also encompasses the process described in steps (a1) to (d1) above further comprising making the compound of Formula 4 by
(e1) reacting a compound of Formula 1

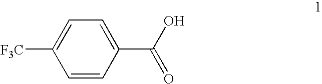

with a second chlorinating agent in the presence of dimethylformamide to yield the acid chloride of Formula 1a

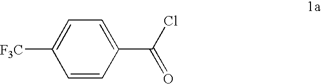

and reacting the compound of Formula 1a with 2,5-dimethylthiophene in the presence of a first Lewis acid reagent or first strong Bronsted acid to yield a compound of Formula 2

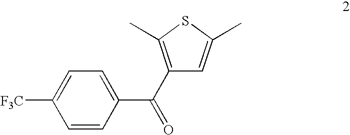

(f1) reacting the compound of Formula 2 with brominating agent in the presence of a zinc salt catalyst to yield a compound of Formula 3

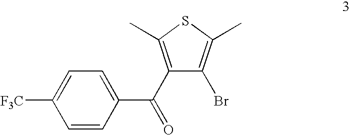

and (g1) reducing the compound of Formula 3 with a silane reducing agent in the presence of a second Lewis acid reagent or second strong Bronsted acid to yield a compound of Formula 4.

For the above step (e1) to (g1), the following amounts of the reagents may be used (relative to the first reagent in the process step): 1 to 2 equivalents of the second chlorinating agent, 0.01 to 0.1 equivalents of dimethylformamide, 0.8 to 1.5 equivalents of 2,5-dimethylthiophene, 1 to 2 equivalents of the first Lewis acid reagent or first strong Bronsted acid, 0.5 to 2 equivalents of the brominating agent, 0.01 to 0.2 equivalents of the zinc salt catalyst, 1 to 10 equivalents of the silane reducing agent, and 1 to 100 equivalents of the second Lewis acid reagent or second strong Bronsted acid.

The terms “first Lewis acid reagent” and “second Lewis acid reagent” independently mean an electron pair acceptor. Examples of such reagents include aluminum chloride, boron trifluoride, boron trichloride, aluminum bromide, iron(III) chloride, niobium pentachloride, ytterbium(III) triflate, titanium tetrachloride and the like. In an embodiment of the invention the first Lewis acid reagent and second Lewis acid reagent are titanium tetrachloride. The terms “first strong Bronsted acid” and “second strong Bronsted acid” independently mean a compound that donates a hydrogen ion to another compound for example trifluoroacetic acid, sulfuric acid, hydrogen fluoride, phosphoric acid and trifluoromethanesulfonic acid.

The term “brominating agent” means a compound capable of introducing bromine into a molecule. Examples include Br₂, phosphorus tribromide, bromine chloride, and aluminum tribromide. In an embodiment of the invention the brominating agent is Br₂.

The term “zinc salt catalyst” means a salt of zinc that acts as a Lewis acid. Examples include zinc nitrate, zinc chloride, zinc carbonate, zinc bromide, zinc fluoride, zinc hydroxide, zinc sulfate, zinc iodide and zinc oxide or mixtures thereof In an embodiment of the invention the zinc salt catalyst is ZnCl₂.

The term “silane reducing agent” means a silane compound capable of reducing a carbonyl substrate. Examples include trialkylsilanes, dialkylsilanes or trialkoxysilanes. More specific examples include dimethylsilane, diethylsilane, trimethoxysilane and triethoxysilane. In an embodiment of the invention the silane reducing agent is Et₃SiH.

The invention also encompasses the process described in steps (a1) to (c1) above further comprising making the compound of Formula 7 by
(h1) reacting a compound of Formula 6

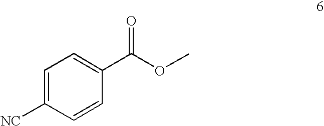

with an ethyl Grignard reagent of the formula EtMgX, wherein X is a halide, in the presence of titaniumisopropoxide followed by a boron trihalide to yield a compound of Formula 7.

For the above step (h1), the following amounts of the reagents may be used (relative to the first reagent in the process step): 2 to 4 equivalents of the ethyl Grignard reagent, 1 to 2 equivalents of titaniumisopropoxide, and 1 to 4 equivalents of boron trihalide.

Examples of an ethyl Grignard reagent include ethyl magnesium bromide and ethyl magnesium chloride. In an embodiment of the invention the Grignard reagent is EtMgBr.

The term “boron trihalide” means BX₃, wherein X is F, Cl or Br, or an adduct thereof such as with an ether. In an embodiment of the invention the boron trihalide is boron trifluoride diethyl ether.

The invention also encompasses the process described in steps (a1) to (d1) above further comprising making the compound of Formula 4 by
(i1) reacting 2,5-dimethylthiophene with a compound of Formula 11

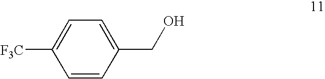

in the presence of a first transition metal salt reagent and a strong acid to yield a compound of Formula 12

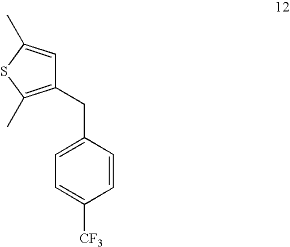

and (j1) reacting the compound of Formula 12 with brominating agent in the presence of a zinc salt catalyst to yield a compound of Formula 4.

For the above steps (i1) to (j1), the following amounts of the reagents may be used (relative to the first reagent in the process step): 0.5 to 2 equivalents of compound 11, 0.1 to 1 equivalents of the first transition metal salt reagent, 0.1 to 1 equivalents of the strong acid, 0.5 to 2 equivalents of the brominating agent and 0.01 to 0.2 equivalents of the zinc salt catalyst.

The term “first transition metal salt reagent” means the salt of a transition metal that acts as a Lewis acid. Examples include CoCl₂, CuBr, CuCl, CuBr₂, CuCl₂, FeCl₂, Fe(OAc)₂, [Fe(acetylacetone)₃], FeCl3, Fe(ClO₄)₃, Fe(BF₄)₂, MnO₂, MnCl₂, MnSO₄, ZnCl₂, Zn(OAc)₂, including hydrates thereof. Preferred are iron(III) salts. In an embodiment of the invention the first transition metal reagent is FeCl₃.

The term “strong acid” means for example a sulfonic acid, preferably methylsulfonic acid, which is an embodiment of the invention.

The terms “brominating agent” and “zinc salt catalyst” are as previously defined.

The invention encompasses a process for synthesizing a compound of Formula I

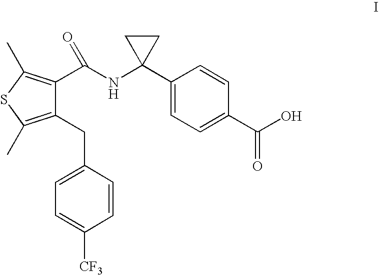

or a pharmaceutically acceptable salt thereof, comprising:
(a2) reacting a compound of Formula 12

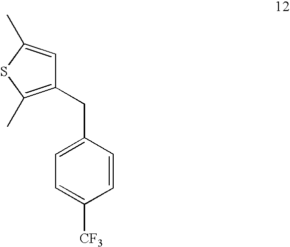

with a compound of Formula 13

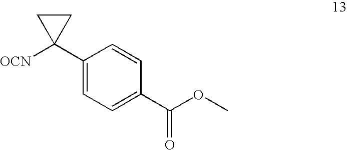

in the presence of a first transition metal salt reagent to yield a compound of Formula 8

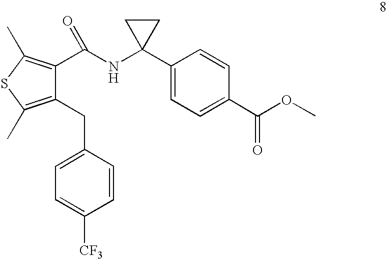

(b2) hydrolyzing the compound of Formula 8 with a strong base of formula X¹—OH or X²—(OH)₂, wherein X¹ is selected from the group consisting of: potassium, cesium, lithium, sodium and rubidium, and X² is selected from the group consisting of: barium, strontium and calcium, followed by acidification to yield the compound of Formula I; and
(c2) optionally reacting the compound of Formula I with a base to yield a pharmaceutically acceptable salt of the compound of Formula I.

For the above steps (a2) to (c2), the following amounts of the reagents may be used (relative to the first reagent in the process step): 0.8 to 1.5 equivalents of compound 13, 0.5 to 2 equivalents of the first transition metal salt catalyst, 1 to 10 equivalents of the strong base, 1 to 10 equivalents of the acid used in the acidification step, and 1 to 1.5 equivalents of the base used to form the pharmaceutically acceptable salt.

The terms “first transition metal salt reagent,” “acidification” and “base” are as previously defined.

The invention also encompasses the process of steps (a2) to (c2) above further comprising making compound of Formula 12 by
(d2) reacting 2,5-dimethylthiophene with a compound of Formula 11

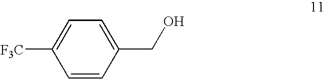

in the presence of a second transition metal salt reagent and a strong acid to yield a compound of Formula 12.

For the above step (d2), the following amounts of the reagents may be used (relative to the first reagent in the process step): 0.5 to 2 equivalents of compound 11, 0.1 to 1 equivalents of the second transition metal salt reagent and 0.1 to 1 equivalents of the strong acid.

The term “second transition metal salt reagent” means the same as “first transition metal salt reagent” but is independent of such definition. In an embodiment of the invention the first transition metal reagent is FeCl₃.

The term “strong acid” is as previously defined.

The invention also encompasses the process described in steps (a2) to (c2) above further comprising making the compound of Formula 13 by
(e2) reacting a compound of Formula 7

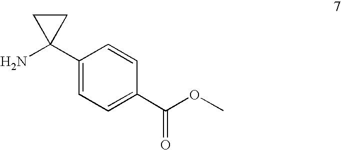

with COCl₂ in the presence of an amine base to yield the compound of Formula 13.

For the above step (d2), the following amounts of the reagents may be used (relative to the first reagent in the process step): 1 to 2 equivalents of COCl₂ and 1 to 2 equivalents of the amine base.

The term “amine base” is as previously defined.

Unless specified, all reactions may be conducted in an appropriate solvent which can be readily selected by one having ordinary skill in the art in view of the examples that follow.

The invention also encompasses the diethylamine salt of the compound of Formula I

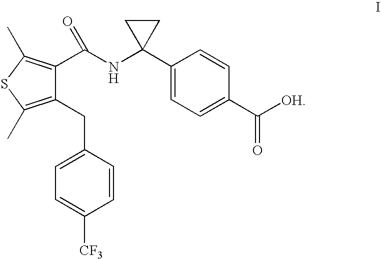

The following abbreviations have the indicated meanings:
DIPEA=N,N′-diisopropylethylamine
Et=ethyl
DCE=dichloroethane
DMF=dimethylformamide
HATU=2-(1H-7-azabenzotriazol-1-yl)-1,1,3,3-tetramethyl uronium hexafluorophosphate methanaminium
Me=methyl
Ms=mesyl
MTBE=methyl t-butyl ether
NBS=N-bromosuccinimide
Ph=phenyl
TFA=trifluoroacetic acid
THF=tetrahydrofuran
TMEDA=tetramethylethylenediamine

### Examples

### Example A

### 4-{1-[({2,5 -dimethyl-4-[4-(trifluoromethyl)benzyl]-3-thienyl}carbonyl)amino]cyclopropyl}benzoic acid diethylamine salt

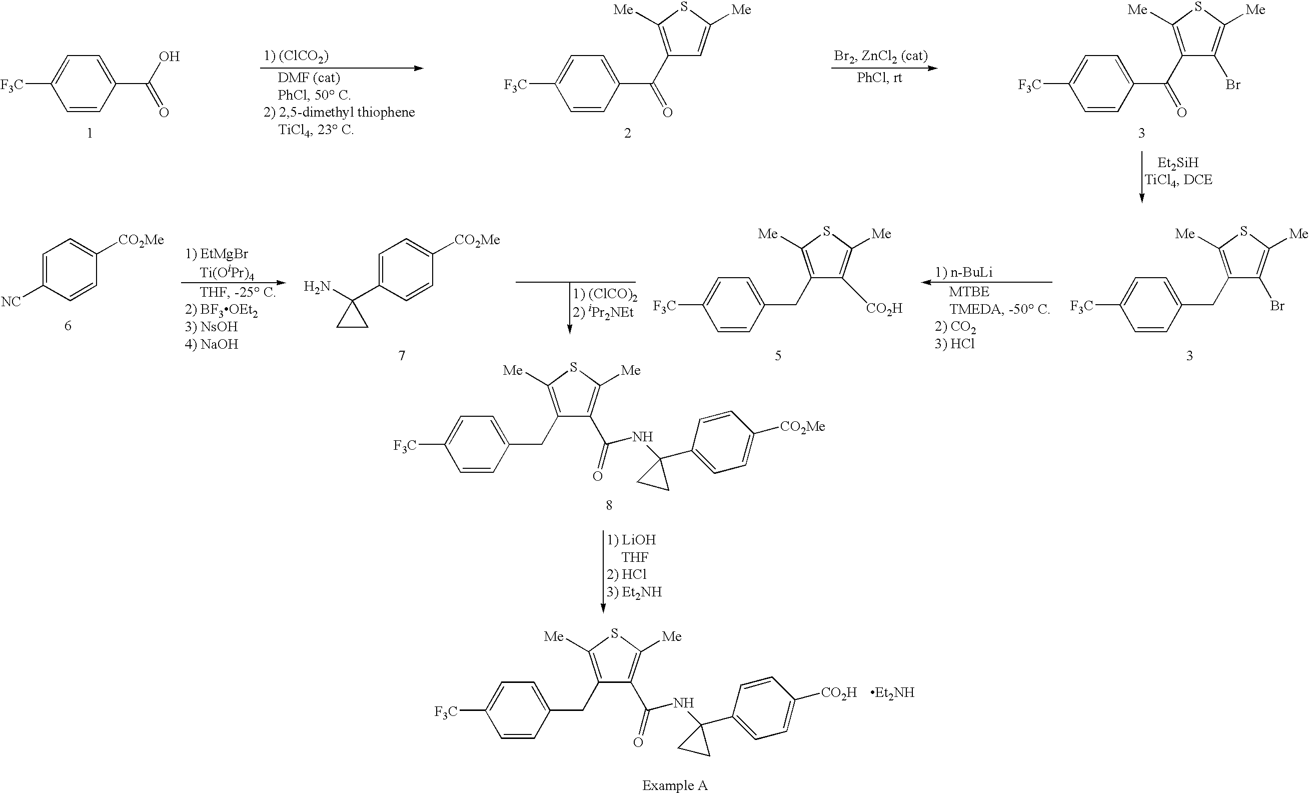

### Step 1—Cyclopropanation

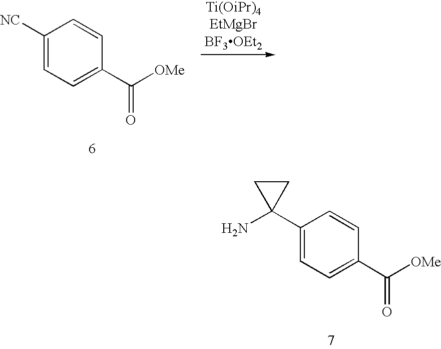

| Materials | MW | Amount | Moles | Eq |
|---|---|---|---|---|
| Methyl 4-cyanobenzoate 6 | 161.16 | 2.60 Kg | 16.13 | 1.00 |
| Ti(OiPr)₄ | 284.22 | 4.73 L | 16.13 | 1.00 |
| EtMgBr [3.07 M] | 133.27 | 10.51 L | 32.27 | 2.00 |
| BF₃•OEt₂ | 141.93 | 4.09 L | 32.27 | 2.00 |
| Toluene [15 mL/g] | | 40 L | | |
| 2-Me-THF [30 mL/g | | 80 L | | |
| 3 N HCl [15 mL/g] | | 40 L | | |
| 3 N NaOH [10 mL/g] | | 26 L | | |

A visually clean 100 L 5-neck round-bottom flask equipped with a mechanical stirrer, a thermocouple, a nitrogen inlet was charged with the nitrile-ester 6 (2.60 Kg, 1.00 eq) and toluene (40 L, 15 mL/g). The mixture was cooled to −25° C. using a cooling bath filled with 2-propanol and dry ice. The Ti(OiPr)₄ (4.73 L, 1.00 eq) was added to the solution over 5 minutes. The ethylmagnesium bromide (10.5 L, 2.0 eq) was added over a period of 2 hrs keeping the temperature of the reaction mixture between −25° C. and −13° C. The mixture was aged at −20° C. for 30 minutes. The borontrifluoride diethyl ether (4.09 L) was added over 40 minutes keeping the reaction mixture between −24° C. and −8° C. The mixture was aged at −20° C. for 30 minutes, then the conversion was measured by HPLC and showed to be 93%. The reaction was quenched by the addition of HCl. 20 L (7.5 mL/g) of 3N HCl was slowly added (over 30 minutes) to the reaction mixture causing an exotherm of 39° C. (exotherm −16° C.→+23° C.). The organic layer was transferred to the extractor, then the rest of the HCl (20 L, 7.5 mL/g) was added to the flask to dissolve the amine salt. After stirring for 10 minutes, the aqueous layer was transferred to the extractor. The mixture was stirred 10 minutes, then the layers were separated. The aqueous layer was washed with toluene (13 L, 5 mL/g). The aqueous layer was extracted with 2-Me-THF 2×10 mL/g (2×26 L) and 2×5 mL/g (2×13 L). Combined Me-THF layers were washed with 3N NaOH (26 L, 10 mL/g) and the pH of the NaOH solution was adjusted to pH 9 using 10N NaOH (1.6 L) prior to the layer separation. The organic layer was washed with brine (13 L, 5 mL/g). The assay yield of the cyclopropylamine 7 was determined on the Me-THF layer prior to its concentration and showed to be 43.2% (1.334 Kg). The losses to the aqueous layer were bellow 3.8%.

### Step 2—Cycloproylamine, Methanesulfonic Acid Salt Formation

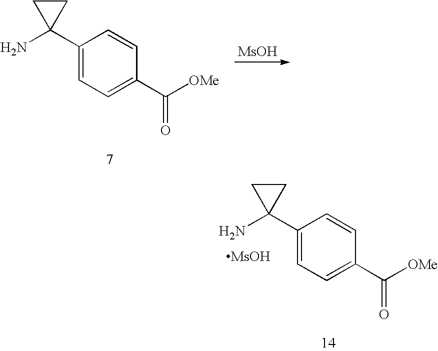

| Materials | MW | Amount | Moles | Eq |
|---|---|---|---|---|
| Cyclopropylamine 7 | 191.23 | 2.63 Kg | 13.75 | 1.00 |
| MsOH | 96.11 | 1.00 L | 15.40 | 1.12 |
| THF [14 mL/g] | | 37 L | | |

A visually clean 100 L 5-neck round-bottom flask equipped with a mechanical stirrer, a thermocouple, a nitrogen inlet and a cooling bath was charged with the cyclopropylamine 7 (2.63 Kg, 1.00 eq) and THF (32 L, 12 mL/g). To the solution was added the MsOH (1.00 L, 1.12 eq) as a THF (4.0 L, 1.5 mL/g) solution over a period of 2 hrs. After the first 10 minutes of addition, seeds (500 mg) were added to start the crystallization. The solution was stirred at RT for a period of 15 hrs. The suspension was filtered and rinsed with a small portion of the mother liquors. The salt was washed twice with cold THF (2×8 L, 2×3 mL/g), then dried on the frit for 3 hrs. The salt was dried in the vacuum oven first at 30° C. for 20 hrs, then at 50° C. for a period of 60 hrs. The yield of material obtained was 3.93 Kg, which was 94.4% wt (yield=92.9%). The losses to the mother liquors were 8.2 g (0.3%).

### Step 3—Methanesulfonic Acid Salt Break

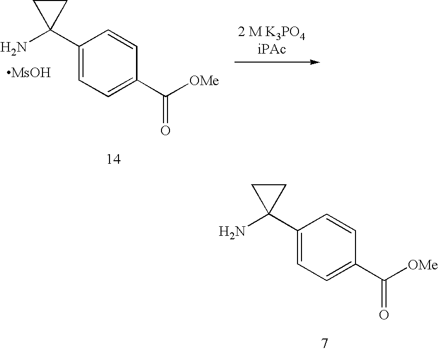

| Materials | MW | Amount | Moles | Eq |
|---|---|---|---|---|
| MsOH salt 14 (94.4% wt) | 287.33 | 3.93 Kg | 12.91 | 1.00 |
| 2 M K₃PO₄ [5 mL/g] | | 19 L | | |
| iPAc [10 mL/g] | | 39 L | | |

A visually clean 160 L 5-neck extractor equipped with a mechanical stirrer, a thermocouple and a nitrogen inlet was charged with the MsOH salt 14 (3.85 Kg, 1.00 eq) and iPAc (39 L, 10 mL/g). To the solution was added the 2M K₃PO₄ (19 L, 5 mL/g). The solution was stirred at RT for a period of 2 hrs to completely break the salt so that no solid remained in suspension. The layers were separated. The organic layer was washed once with water (19 L, 5 mL/g) and once with saturated NaCl solution (19 L, 5 mL/g). The assay yield of cyclopropylamine was checked on the iPAc solution and showed to be 2.445 Kg (98.8%). The losses to the aqueous layer were below 0.1%. The iPAc layer was concentrated on a rotavap and flushed with 10 L THF.

### Step 4—Acid Chloride Formation & Freidel-Crafts Acylation.

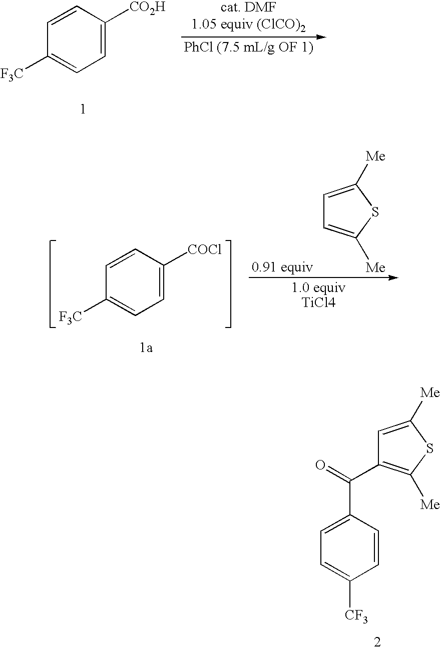

| Materials | MW | Amount | Moles |
|---|---|---|---|
| Benzoic Acid 1 | 190.06 | 5.96 kg | 31.4 (1.00 eq) |
| Oxalyl chloride | 126.93 (d 1.455) | 2.87 L | 32.9 (1.05 eq) |
| DMF | | 10 mL | |
| Chlorobenzene | | 45.0 L | |
| 2,5-Dimethylthiophene | 112.19 (d 0.985) | 3.25 L | 28.5 (0.91 eq) |
| Titanium (IV) chloride | 189.71 (d 1.73) | 3.44 L | 31.4 (1.00 eq) |
| 1 N HCl | | 60 L | |
| Heptane | | 40 L | |
| Half-Brine | | 20 L | |

A visually-clean, 100 L 5-neck round-bottom flask was fitted with mechanical stirrer, reflux-condenser, internal temperature probe, nitrogen inlet was connected to a scrubber filled with 20-litres of 5N NaOH. The flask was charged with chlorobenzene, benzoic acid 1 and oxalyl chloride, then heated with a steam bath until the internal temperature reached 50° C. DMF was then added dropwise.

A vigorous evolution of gas was observed upon addition of DMF. The steam bath was turned off after 20 minutes, and the reaction maintained an internal temperature of 45-50° C. After 1 hr, the cloudy reaction mixture was assayed by HPLC of an aliquot, which indicated 96% of acid 1 to acid chloride la.

After the internal temperature had dropped to 22° C., dimethylthiophene was added to the reactor at once, followed by titanium (IV) chloride over 1 h via the addition funnel.

The internal temperature was observed to raise to a maximum of 36° C. during addition of titanium (IV) chloride. The crude mixture was allowed to cool to room temperature overnight.

A visually-clean 160-litre extractor was charged with 1N HCl. The crude reaction mixture was transferred into the extractor (An internal temperature probe indicated the reaction mixture temperature to vary from 22° C. to 34° C.) with vigorous stirring. After 5 min of vigorous stirring, the phases were allowed to separate. The organic layer (bottom) was removed and the aqueous layer back-extracted with heptane. The organic phases were combined, washed with half-brine then filtered through a 20 micron filter into a visually-clean 100 L round-bottom flask which was fitted with mechanical stirrer and connected to a batch concentrator. Solvent was removed under vacuum to afford a thin brown oil.

After the material had been concentrated to 15.61 kg of thin brown oil, and aliquot was removed for HPLC analysis, which determined the material to be 52.77 wt % ketone 2, or 8.24 kg, a 92.4% assay yield.

It should be noted that the reaction is easier (and safer, particularly on scale) if the acid and catalytic DMF are mixed first and the oxalyl chloride is added slowly to control the rate of gas evolution.

### Step 5—Bromination.

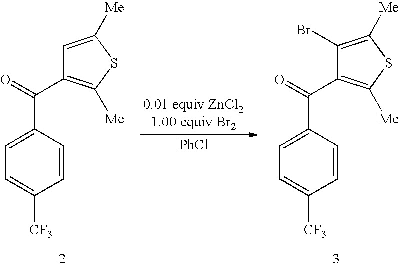

| Materials | MW | Amount | Moles |
|---|---|---|---|
| Ketone 2 (52.77 wt %) | 284.05 | 13.27 kg | 24.7 (1.00 eq) |
| Zinc Chloride | 136.28 | 33.6 g | 0.25 (0.01 eq) |
| Bromine | 159.8 (d 3.11) | 3.94 kg | 24.7 (1.00 eq) |
| Chlorobenzene | | 33.0 L | |
| 1 N HCl | | 45.0 L | |
| Heptane | | 25.0 L | |
| Half-Brine | | 20.0 L | |

A visually-clean, 100 L 5-neck round-bottom flask was fitted with mechanical stirrer, addition funnel, internal temperature probe, nitrogen inlet and connected to a scrubber filled with 20-litres of 5N NaOH. The flask was charged with ketone 2, chlorobenzene, and zinc chloride, then cooled via an external ice-water bath until the internal temperature reached 16° C. Bromine was charged to the addition funnel, then added over 1 h.

The internal temperature was observed to rise to a maximum of 26° C. during addition of bromine. The mixture was vigorously stirred for 15 minutes after the addition was complete.

A visually-clean 160-litre extractor was charged with 1N HCl. The crude reaction mixture was transferred into the extractor (internal temperature probe indicated the reaction mixture temperature to vary from 22° C. to 34° C.) with vigorous stirring. After 5 min of vigorous stirring, the phases were allowed to separate. The organic layer (bottom) was removed and the aqueous layer back-extracted with heptane. The organic phases were combined, washed with half-brine then transferred into a visually-clean 100 L round-bottom flask which was fitted with mechanical stirrer and connected to a batch concentrator. Solvent was removed under vacuum, with a 40-L heptane flush, to afford a thin brown oil.

After the material had been concentrated to 10.29 kg of thin brown oil, and aliquot was removed for HPLC analysis, which determined the material to be 80.0 wt % bromoketone 3, or 8.35 kg, a 93.6% assay yield.

### Step 6—Reduction.

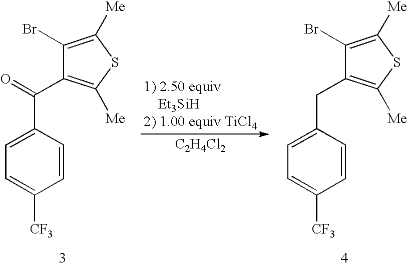

| Materials | MW | Amount | Moles |
|---|---|---|---|
| Bromoketone 3 | 363 | 10.44 kg | 23.1 (1.00 eq) |
| (80.0 wt %) | | | |
| Triethysilane | 116.28 (d 0.728) | 6.70 kg | 57.7 (2.50 eq) |
| Titanium (IV) chloride | 189.71 (d 1.73) | 2.53 L | 23.1 (1.00 eq) |
| Dichloroethane | | 34.0 L | |
| 1 N HCl | | 42.0 L | |
| Heptane | | 20.0 L | |
| Water | | 20.0 L | |
| Silica gel | | 16.0 kg | |
| Toluene | | 40 L | |

A visually-clean, 100 L 5-neck round-bottom flask was fitted with mechanical stirrer, addition funnel, internal temperature probe, nitrogen inlet and outlet. The flask was charged with bromoketone 3, triethylsilane and dichloromethane, then cooled via an external isopropanol/CO₂ bath until the internal temperature reached −1° C. Titanium (IV) chloride was charged to the addition funnel, then added over 1 h.

The internal temperature was observed to raise to a maximum of 30° C. during addition of titanium (IV) chloride. The exotherm continued after addition was complete, to a maximum internal temperature of 43° C. over 0.5 h. The mixture was stirred an additional 2 h, during which time the temperature dropped to 8° C.

A visually-clean 160-litre extractor was charged with 1N HCl. The crude reaction mixture was transferred into the extractor (internal temperature probe indicated the reaction mixture temperature to vary from 22° C. to 34° C.) with vigorous stirring. After 5 min of vigorous stirring, the phases were allowed to separate. The organic layer (bottom) was removed and the aqueous layer back-extracted with heptane. The organic phases were combined and washed with water.

In two 40-L portions, the crude organic phase was transferred into a visually-clean 100 L round-bottom flask which was fitted with mechanical stirrer, and stirred over 4 kg of silica. After stirring for 1 h, the material was filtered over a glass frit, washing with heptane (5 L). The filtered crude organic was then transferred into a visually-clean 100 L round-bottom flask and connected to a batch concentrator. Solvent was removed under vacuum, with heating, with a 40-L toluene flush, followed by a 40-L heptane flush, to afford a thin brown oil. Heptane (40 L) and silica gel (8 kg) were added to the reaction flask, and the material was stirred under nitrogen for 72 h. The slurry was filtered over a glass frit, washing with heptane (15 L). The filtered crude organic was then transferred into a visually-clean 100 L round-bottom flask and connected to a batch concentrator. Solvent was removed under vacuum with heating, to afford a thin brown oil.

After the material had been concentrated to 8.31 kg of thin brown oil, and aliquot was removed for HPLC analysis, which determined the material to be 36.30 wt % bromoalkane 4, or 3.02 kg, a 37.6% assay yield.

The low yield in this step was due to polymerization of the reduction product. The undesired side reaction could be avoided by carefully lowering the amount of residual chlorobenzene from the bromination step to <1%. This was achieved by flushing the crude bromination mixture with toluene prior to solvent switching into 1,2-dichloroethane for the ketone reduction. This reaction was been re-run on a 1 Kg scale using this protocol and proceeded in 84% yield

### Step 7—Metal-Halogen Exchange and Acid Formation.

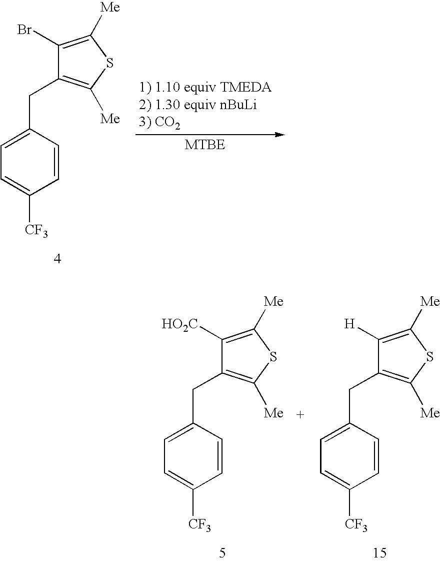

| Materials | MW | Amount | Moles |
|---|---|---|---|
| Bromoalkane 4 | 347.98 | 4.00 kg | 4.31 (1.00 eq) |
| (37.6 wt %) | | | |
| Tetramethyl- | 116.21 (d 0.775) | 711 mL | 4.74 (1.10 eq) |
| ethylenediamine | | | |
| nBuLi (2.5 M in | | 2.24 L | 5.60 (1.30 eq) |
| hexanes) | | | |
| MTBE | | 20.0 L | |
| CO₂ (dry gas) | | ~300 g | |
| 1 N HCl | | 13.0 L | |
| MTBE | | 8.0 L | |
| 0.5 N KOH | | 19.5 L | |
| 6 N HCl | | 1.25 L | |
| MTBE | | | |
| Half-brine | | | |
| Heptane | | | |

A visually-clean, 50 L 5-neck round-bottom flask was fitted with mechanical stirrer, addition funnel, internal temperature probe, nitrogen inlet and outlet. The flask was charged with bromoalkane 4, tetramethylethylenediamine and MTBE, then cooled via an external isopropanol/CO₂ bath until the internal temperature reached −65° C. nBuLi was charged to the addition funnel, then added over 1 h.

The internal temperature was observed to rise to a maximum of −58° C. during addition of nBuLi. The mixture was stirred an additional 0.5 h, during which time the temperature dropped to −62° C.

Gaseous CO₂ was bubbled into the reaction mixture, over 1.5 h. A 16-gauge, 100 cm-long needle was used to ensure that the reagent was delivered below the surface of the reaction mixture.

The internal temperature was observed to rise to a maximum of −54° C. during addition of CO₂. After 1.5 h, the internal temperature dropped to −60° C., and an aliquot was taken from the crude mixture. HPLC analysis indicated ˜85% CO₂ incorporation (vs reduction).

The cooling-bath was replaced with a warm-water bath until the internal temperature reached −25° C.; then 1N HCl was added to the reactor. After vigorously stirring for 5 min, the biphasic solution was transferred into a visually-clean 100-L extractor with vigorous stirring. After 5 min of vigorous stirring, the phases were allowed to separate. The aqueous layer (bottom) was removed and the organic layer collected. The aqueous layer was back-extracted with MTBE (6 L). The organic phases were combined and treated with 0.5N KOH (13.0 L), with vigorous stirring for 5 minutes. After the layers were allowed to separate, the aqueous layer was collected. The organic phase was re-extracted with 0.5N KOH (6.5 L) and the aqueous layers was collected. After removal of the organic phase, the combined aqueous layers were returned to the extractor which was also charged with MTBE (23 L). The biphasic solution was acidified by addition of 6N HCl (1.25 L) until pH ˜1, and the biphasic solution vigorously stirred for 10 min.

After the layers were allowed to separate, and the organic layer was collected and washed with half-brine (13 L). The crude organic material was concentrated in vacuo on the rotovap, flushing with heptane (10 L) to afford a yellow solid (˜4.5 kg).

The crude solid was charged to a visually-clean, 25-L round-bottom flask which was fitted with mechanical stirrer, internal temperature probe, nitrogen inlet and outlet. The flask was charged with crude acid 6 and heptane, then cooled via an external ice/water bath until the internal temperature reached 2° C. The slurry was vigorously stirred for 6 h, then filtered over a glass-frit, washing with cold heptane (1.25 L). The filter cake was dried via house-vacuum under nitrogen overnight. The pale yellow solid was transferred to vacuum-oven and dried at 50° C. for 24 h.

A total of 1.22 kg dry yellow solid was collected. HPLC analysis indicated the material to be 87 wt % acid 5, or 1.06 kg, 79% assay yield.

### Step 8—Amidation/Hydrolysis

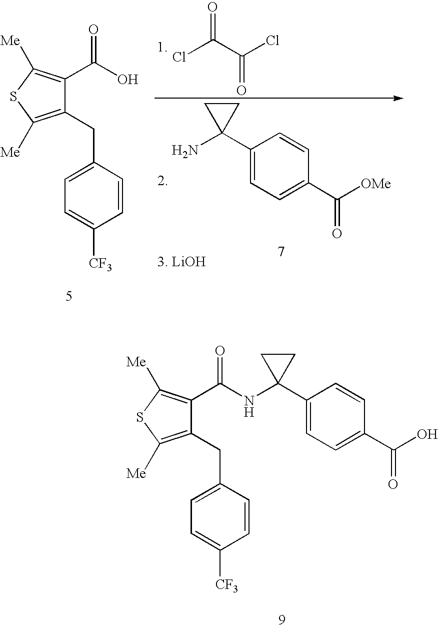

| Materials | MW | Amount | Moles | Eq |
|---|---|---|---|---|
| Thiophene acid 5 | 314.32 | 2.68 Kg | 8.54 | 1.00 |
| Oxalyl chloride | 126.93 | 897 mL | 10.25 | 1.20 |
| DMF | 73.09 | 6.64 mL | 0.085 | 1% |
| Cyclopropylamine 7 | 191.23 | 1.88 Kg | 9.82 | 1.15 |
| N,N-diisopropylethylamine | 129.25 | 2.24 L | 12.81 | 1.50 |
| LiOH 4 N | 23.95 | 7.47 L | 29.9 | 3.50 |
| THF [12 mL/g] | | 32 L | | |
| MeOH [4 mL/g] | | 10.7 L | | |
| 2 N HCl [7 mL/g] | | 19 L | | |
| Me-THF [25 mL/g] | | 67 L | | |

A visually clean 100 L 5-neck round-bottom flask equipped with a mechanical stirrer, a thermocouple, a nitrogen inlet, a cooling bath and a NaOH scrubber was charged with the thiophene acid 5 (2.95 Kg at 91% wt=2.68 Kg, 1.00 eq) and THF (16 L, 6 mL/g). The DMF (6.64 mL, 1% mol) was added. The oxalyl chloride (897 mL, 1.20 eq) was added to the solution over a period of 30 minutes at RT. An exotherm of 10° C. was noticed during the addition of the oxalyl chloride (temperature rose from 17° C. to 27° C.). The mixture was aged at RT for 2 hrs (conversion 99.9%), then the solvent and excess oxalyl chloride were removed using the batch concentrater. The residue was flushed with THF (20 L). The residue was dissolved in THF (27 L, 10 mL/g) and the solution was cooled to 3° C. Diisopropylethylamine (2.24 L, 1.50 eq) was added to the solution. The cyclopropylamine 7 (1.88 Kg, 1.15 eq) was added to the solution as a THF solution (5 L, 2 mL/g) over a period of 30 minutes. An exotherm of 20° C. was observed (temperature 7° C.→27° C.). The mixture was aged 30 minutes. The conversion to the amide-ester was 99.8%. To the solution was added MeOH (4 mL/g, 10.7 L) and the 4N LiOH (7.47 L, 3.5 eq). An exotherm of 14° C. was observed (temperature 17° C.→31° C.). The mixture was heated to 55° C. and kept at this temperature for 1.5 hrs. The conversion to the amide-acid was 99.5%. The mixture was cooled to 22° C. and the reaction was quenched by the addition of 2N HCl (19 L, 7 mL/g). The organic solvents were removed using the batch concentrator and flushed with 20 L of Me-THF. The residue (as a suspension in HCl) was dissolved in Me-THF (54 L, 20 mL/g). The biphasic mixture was transferred to the extractor and the layers were separated. The aqueous layer was back extracted using Me-THF (13 L, 5 mL/g). The combined organic layers were washed with water (13 L, 5 mL/g). The assay yield of the compound 9 was determined in the organic layer prior to its concentration and shown to be 88.0% (3.56 Kg). The losses to the aqueous layer were below 0.1%.

### Step 9—Et₂NH Salt Formation

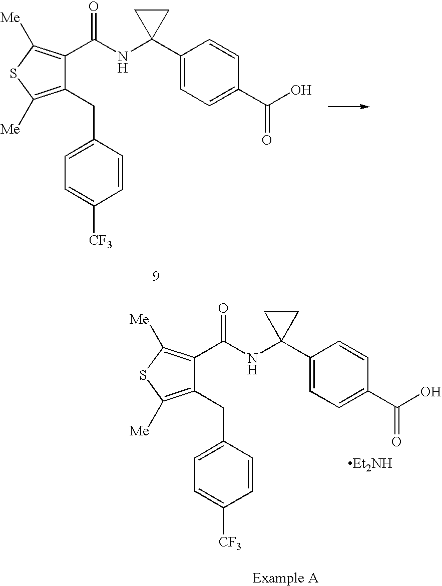

| Materials | MW | Amount | Moles | Eq |
|---|---|---|---|---|
| Compound 9 | 473.51 | 3.54 Kg | 7.48 | 1.00 |
| Et₂NH | 73.14 | 1.18 L | 11.41 | 1.52 |
| Examples A seeds | 546.64 | 35 g | 0.074 | 1% |
| THF [6 mL/g] | | 21 L | | |
| MTBE [12 mL/g] | | 52 L | | |

The Me-THF solution from the amidation/hydrolysis sequence was passed through a pad of Solka Floc (1.20 Kg) and rinsed with 4 L of THF. The filtrate was transferred to a visually clean 100 L 5-neck round-bottom flask equipped with a mechanical stirrer, a thermocouple, a nitrogen inlet, a heating steam bath and a batch concentrator. The solvent was removed under reduced pressure and the residue was flushed with THF (30 L). The residue was suspended in THF (21 L, 6 mL/g) and the Et₂NH (1.18 L, 1.52 eq) was added to the suspension. A 6° C. exotherm was observed (21° C.→27° C.). The salt dissolved into THF. The mixture was aged 1 hr at RT and the solution was cooled to 22° C. using cooled water. Example A seeds (30.0 g) were added and the mixture was aged 1 hr. MTBE (25 L) was added over 2 hrs, then the suspension was aged 13 hrs at room temperature. The mixture was cooled to 3° C. and more MTBE (13 L, 4 mL/g) was added over 1 hr. The losses to the mother liquors were checked and showed to be ˜22%. MTBE (2×7 L, 2×2 mL/g) was added over 1 hr, the mixture was aged 1.5 hrs, then the mixture was filtered. The cake was rinsed with 1 x 7 L MTBE/THF (2/1) and 2×7 L MTBE. The whole filtration took 5 hrs. The cake was dried on the frit for 62 hrs under nitrogen. Compound A was dried in the vacuum oven at 60° C. for 20 hrs. The yield of Example A was 3.76 Kg (92%) as a beige solid. The purity of the material by HPLC was 97.8APC. ¹H NMR showed the presence of ˜3% mol MTBE.

### Step 10—Purification

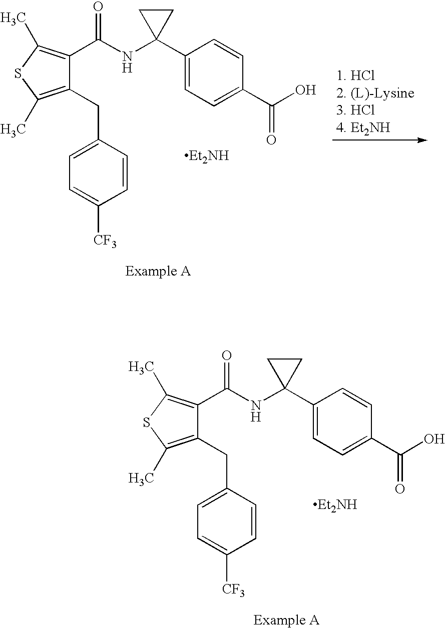

| Materials | MW | Amount | Moles | Eq |
|---|---|---|---|---|
| Example A | 546.64 | 3.67 Kg | 6.714 | 1.00 |
| 1 N HCl | | 40 L | | |
| Me-THF | | 60 L | | |
| (L)-Lysine•H₂O | 164.19 | 1.20 Kg | 7.31 | 1.09 |
| THF | | 74 L | | |
| EtOH | | 1.26 L | | |
| H₂O | | 9.5 L | | |
| Et₂NH | 73.14 | 624 mL | 6.03 | 0.90 |
| Example A seeds | 546.51 | 24 g | 0.074 | 1% |
| MTBE [12 mL/g] | | 29 L | | |

The Example A (3.67 Kg) salt was added to a mixture of Me-THF (30 L) and 1N HCl (20 L, prepared from a 6N HCl solution) and the suspension was stirred at room temperature until complete dissolution (35 min). The layers were separated and the organic layer was washed twice with water (20 L and 10 L). The organic layer was transferred to a visually clean 100 L 5-neck round-bottom flask equipped with a mechanical stirrer, a thermocouple, a nitrogen inlet, a heating steam bath and a batch concentrator. The solvent was removed under reduced pressure and the residue was flushed with THF (20 L).

The residue was dissolved in THF (60 L) and the solution was warmed to 60° C. using a steam bath. A water (9.5 L) solution of the (L)-lysine (1.20 Kg, 1.09 eq) was added over 2 min, followed by the addition of EtOH (1.26 L). The mixture was cooled to 22° C. over 40 min over cold water and ice. The mixture was aged at room temperature for 15 hrs, then filtered and rinsed 3×3 L THF, dried on the frit for 1 hr.

The Compound 9. lysine salt was added to a mixture of Me-THF (30 L) and 1N HCl (20 L, prepared from a 12 N and 6N HCl solution) and the suspension was stirred at room temperature until complete dissolution (40 min). The layers were separated and the organic layer was washed twice with water (20 L and 10 L). The organic layer was transferred via a in-line filter to a visually clean 100 L 5-neck round-bottom flask equipped with a mechanical stirrer, a thermocouple, a nitrogen inlet, a heating steam bath and a batch concentrator. The solvent was removed under reduced pressure and the residue was flushed with THF (20 L).

The residue was suspended in THF (14 L, 6 mL/g) and the Et₂NH (624 mL, 0.90 eq) was added to the suspension. The mixture was aged 30 min at 22° C. then Example A seeds (24.0 g) were added and the mixture was aged 1 hr. MTBE (24 L) was added over 2 hrs, then the suspension was aged 1 hr at room temperature. MTBE (5 L, 2 mL/g) was added over 30 min. The mixture was aged 30 min, then the mixture was filtered. The cake was rinsed with 1×7 L MTBE/THF (2/1) and 2×5 L MTBE. The whole filtration took 4 hrs. The cake was dried on the frit for 8 hrs under nitrogen. The Example A salt was dried in the vacuum oven at 60° C. for 20 hrs. The yield of Example A was 2.78 Kg (75%) as beige solid. The purity of the material by HPLC was 98.7APC. ¹H NMR showed the presence of ˜1.7% mol THF residual.

### Alternate Example A

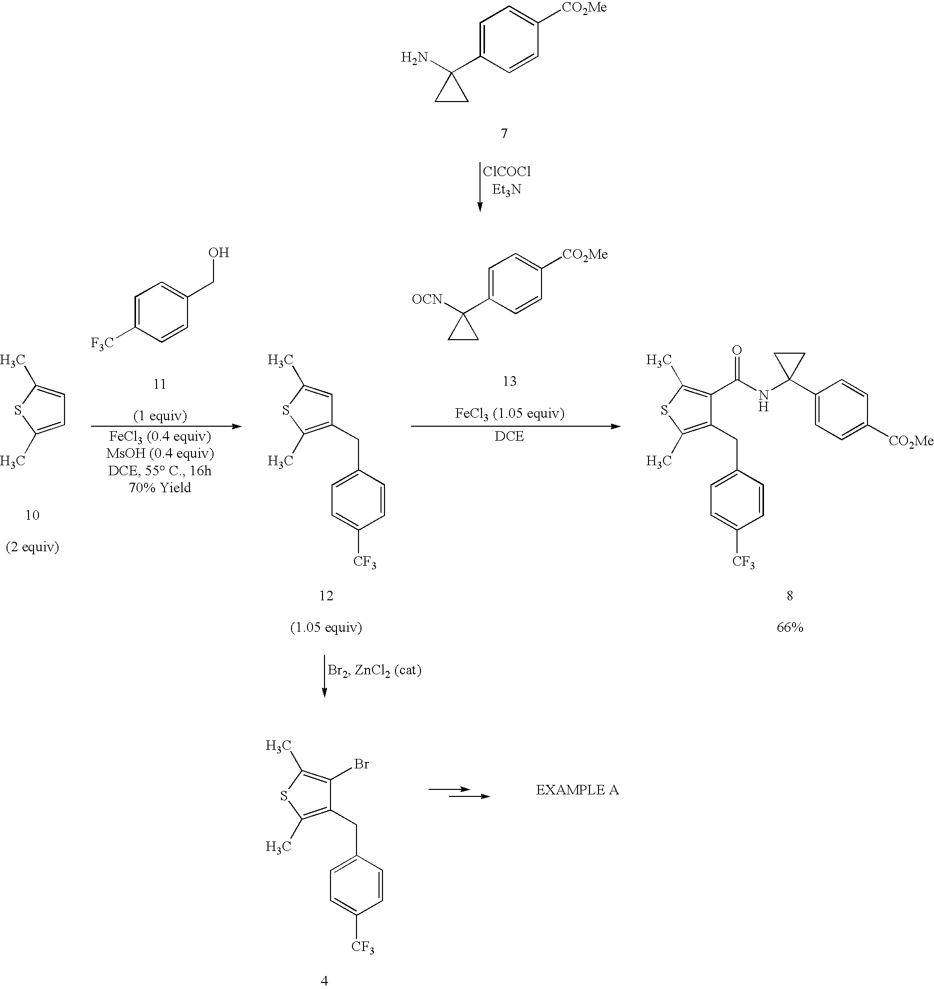

Step 1—Freidel-Crafts Alkylation with 4-Trifluoromethbenzyl Alcohol.

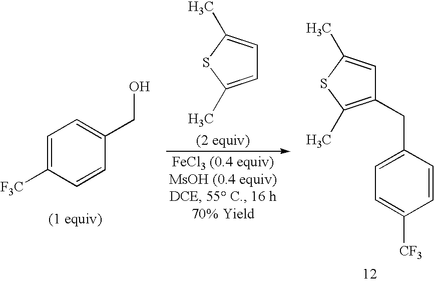

| Materials | MW | Amount | mmoles | Eq |
|---|---|---|---|---|
| 4-Trifluoromethylbenzylalcohol | 176.14 | 257 mg | 1.46 | 1.00 |
| 2,5-Dimethylthiophene | 112.19 | 328 mg | 2.92 | 2.00 |
| FeCl₃ | 162.20 | 95 mg | 0.033 | 0.4 |
| MsOH | 96.11 | 56.1 mg | 0.038 | 0.4 |

The benzylic alcohol was dissolved in DCE (1.2 mL) and the 2,5-dimethylthiophene was added followed by MsOH and FeCl₃. The mixture was warmed to 55° C. and aged 16 h. The reaction was quenched by addition of NH₄Cl solution. The mixture was extracted with MTBE, the organic layer was back extracted once with MTBE and the organic layers were combined, washed with brine, dried over MgSO₄, filtered and concentrated. The assayed yield (relative to an HPLC standard) was 278 mg (70%).

### Step 2—Isocyanate Formation.

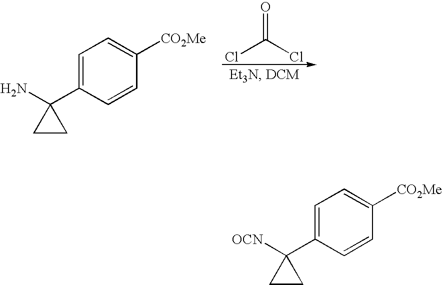

| Materials | MW | Amount | mmoles | Eq |
|---|---|---|---|---|
| Cyclopropyl amine | 191.23 | 6.0 g | 31.4 | 1.00 |
| Triethylamine | 101.19 | 6.98 g | 69.0 | 2.20 |
| Phosgene | 98.92 | 16.29 g | 32.9 | 1.05 |

Phosgene was diluted into DCM (40 mL) and cooled to 0° C. and a DCM (10 mL) solution of cyclopropyl amine and Et₃N was added over 60 min. The mixture was warmed to rt and aged 10 min. The mixture was washed with 1N HCl and brine, then dried over MgSO₄, filtered and concentrated. The residue was purified by flash chromatography (10→30% EtOAc/hexanes) to afford 3.67 g of isocyanate.

### Step 3—Friedel-Crafts Amidation of 12 to Form Ester 8.

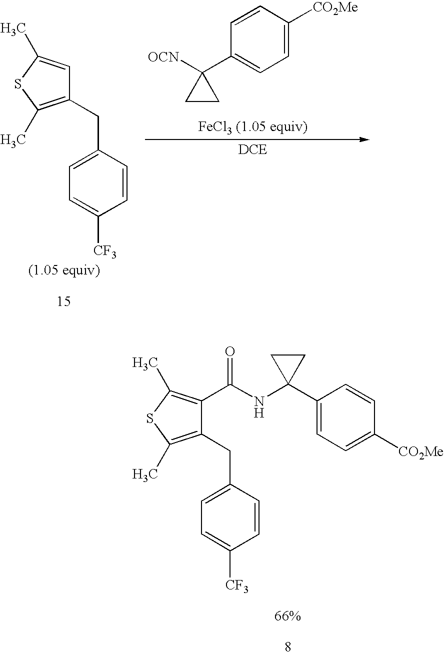

| Materials | MW | Amount | mmoles | Eq |
|---|---|---|---|---|
| Isocyanate | 217.22 | 60 mg | 0.276 | 1.00 |
| Thiophene | 270.31 | 82 mg | 0.304 | 1.10 |
| FeCl₃ | 162.20 | 47 mg | 0.290 | 1.05 |

The thiophene fragment was diluted in DCE (1.5 mL) and the isocyanate was added, followed by FeCl₃. After warming to 70° C. for 15 min the mixture was partitioned between sat^{d} NH₄Cl and 2-MeTHF. The organic layer was washed with brine. The organic layer assayed at 83mg of the desired product (66%).

Example A can be synthesized from the ester 8 as previously described.

The general approach for making the compound of Formula I described in U.S Provisional Application No. 60/837,252, filed on Aug. 11, 2006 is shown in Scheme 3.

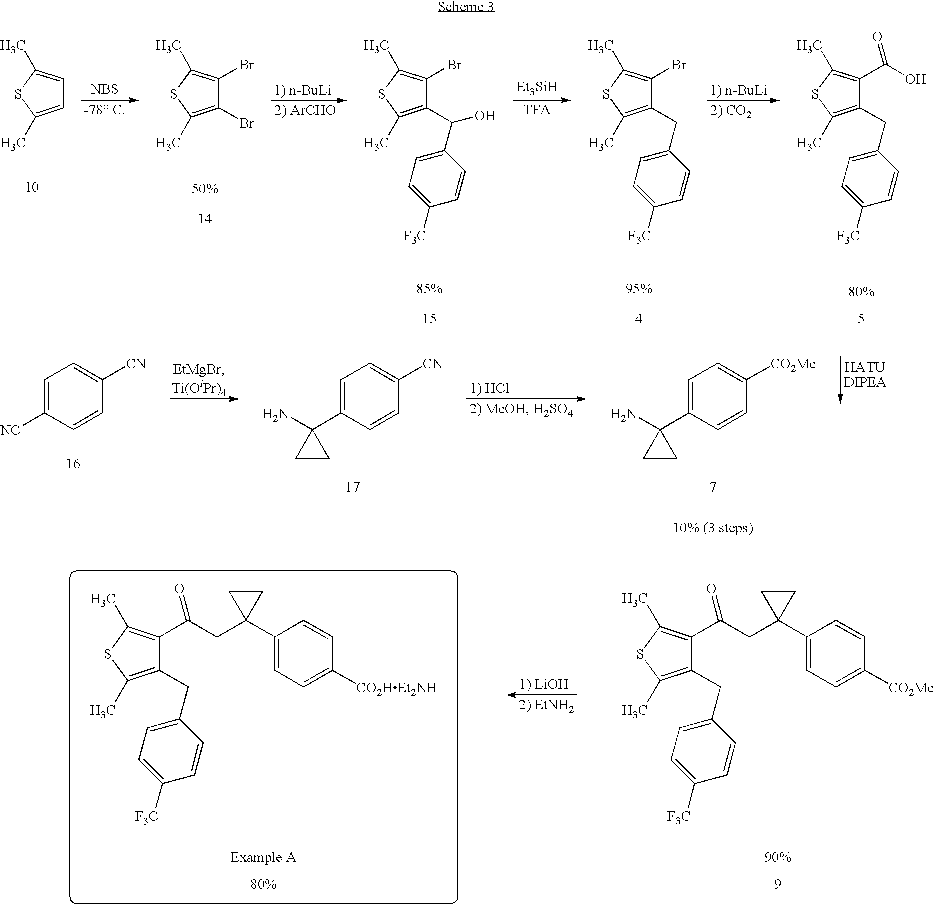

There were number of problems with this route for use in large scale synthesis. The first problem was the dibromothiophene intermediate 14 is formed in low yield and decomposes on standing. Two separate cryogenic steps were required to appropriately functionalize the 3- and 4-positions of the thiophene ring. In the first part of this invention, the use of 14 is obviated by performing a Freidel-Crafts acylation/bromination/ketone reduction sequence which affords bromide 4 without resorting to cryogenic conditions. The second problem is the inefficient, low yielding 3 step sequence used to prepare the cyclopropyl amine from 1,4-dicyanobenzene (10% over 3 steps). This was improved by preparing the amine in a single step from methyl cyano benzoate 6 in 42% yield. The third problem with the prior approach for making the compound of Formula I is the metal halogen exchange/carboxylation sequence. The protocol calls for the use of a mixture of Et₂O and THF as solvent which is problematic on scale in light of the flammability of Et₂O. In the process of the present invention, the transformation was carried out effectively in MTBE when 1 equiv of TMEDA was added to the reaction mixture. Finally, the amidation step in the prior route employed the prohibitively expensive HATU reagent. The invention encompasses a more economically viable coupling protocol which proceeds via the acid chloride derived from 5.

It should also be noted that the free acid of Formula I is poorly bioavailable. The Na, K and NH₄ salts were prepared and found to be weakly crystalline and offered no improvements in pharmacokinetics. It was discovered that both the Et₂NH and L-lysine salts doubled the exposure. The L-lysine salt had an inferior physical stability profile as compared to the Et₂NH salt.

While the first generation approach to the compound of Formula I (Example A) above could be used to prepare multikilogram quantities of the compound of Formula I, there were still opportunities to develop an even more efficient process. A further embodiment of the invention encompasses the use an FeCl₃ mediated benzylation methodology to do an alkylative Freidel-Crafts reaction (Alternative Example A, step 1) in place of the acylation of Example A, which obviates the need for the TiCl₄/Et₃SiH mediated ketone reduction. While this methodology has previously been demonstrated with 2,5-dimethyl thiophene (Iovel, I.; Merlins, K.; Kishel, J.; Zapf, A.; Beller, M. Angew. Chem., Int. Ed. 2006, 44, 3913-3917), this represents the first example of successfully using such an electron deficient benzylic alcohol as a bezylating agent. The invention also encompasses the addition of strong acids (particularly MsOH) resulting in a previously undisclosed acceleratory effect. The first generation approach to the compound of Formula I (Example A) could then be intercepted by bromination of 12 to afford 4. Alternatively, 12 can be amidated directly with isocyanate 13 in the presence of FeCl₃. This second generation approach to the compound of Formula I involves 5 steps in total with a longest linear sequence of 4 steps.

What is claimed is:

1. A process for synthesizing a compound of Formula I 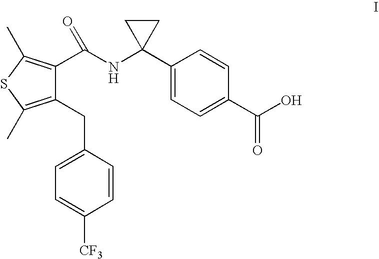
or a pharmaceutically acceptable salt thereof, comprising:
(a1) reacting a compound of Formula 5 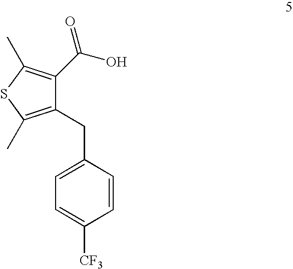
with a first chlorinating agent in the presence of dimethylformamide to yield the acid chloride of Formula 5a 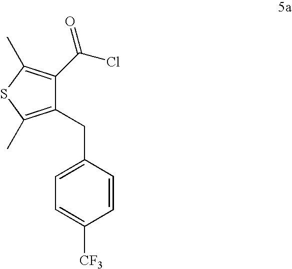
and reacting the compound of Formula 5a with a compound of Formula 7 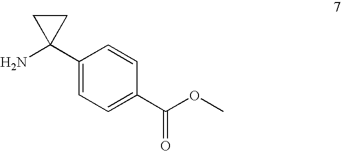
in the presence of an amine base to yield a compound of Formula 8 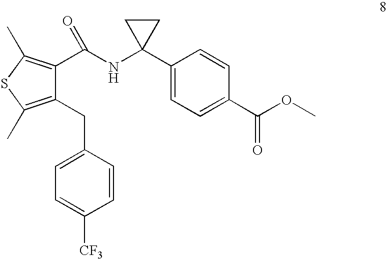
(b1) hydrolyzing the compound of Formula 8 with a strong base of formula X¹—OH or X²—(OH)₂, wherein X¹ is selected from the group consisting of: potassium, cesium, lithium, sodium and rubidium, and X² is selected from the group consisting of: barium, strontium and calcium, followed by acidification to yield the compound of Formula I; and
(c1) optionally reacting the compound of Formula I with a base to yield a pharmaceutically acceptable salt of the compound of Formula I.

2. The process according to claim 1 further comprising making the compound of Formula 5 by
(d1) reacting a compound of Formula 4 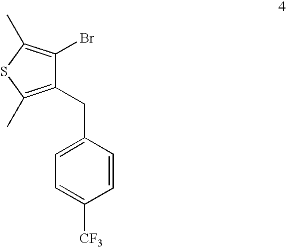
with an organolithium reagent in the presence of tetramethylethylenediamine in a solvent of methyl tertiary-butyl ether at a temperature of at or below about 55° C., and reacting the resulting mixture with CO₂ followed by an acid to yield a compound of Formula 5.

3. The process according to claim 2 further comprising making the compound of Formula 4 by
(e1) reacting a compound of Formula 1 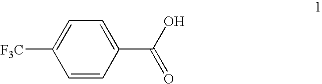
with a second chlorinating agent in the presence of dimethylformamide to yield the acid chloride of Formula 1a 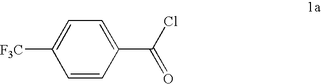
and reacting the compound of Formula 1a with 2,5-dimethylthiophene in the presence of a first Lewis acid reagent or first strong Bronsted acid to yield a compound of Formula 2 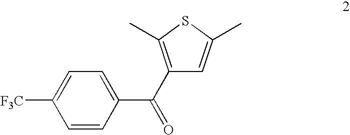
(f1) reacting the compound of Formula 2 with brominating agent in the presence of a zinc salt catalyst to yield a compound of Formula 3 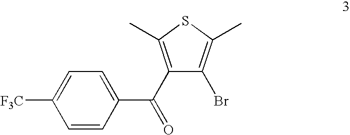
and (g1) reducing the compound of Formula 3 with a silane reducing agent in the presence of a second Lewis acid reagent or second strong Bronsted acid to yield a compound of Formula 4.

4. The process according to claim 1 further comprising making the compound of Formula 7 by
(h1) reacting a compound of Formula 6 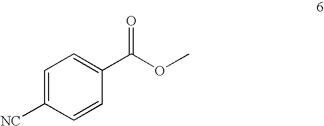
with ethyl Grignard reagent of the formula EtMgX, wherein X is a halide, in the presence of titaniumisopropoxide followed by a boron trihalide to yield a compound of Formula 7.

5. The process according to claim 2 further comprising making the compound of Formula 4 by
(i1) reacting 2,5-dimethylthiophene with a compound of Formula 11 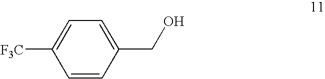
in the presence of a first transition metal salt reagent and a strong acid to yield a compound of Formula 12 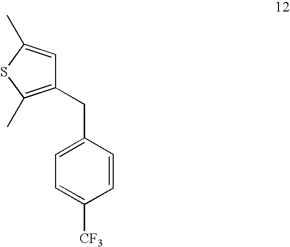
and (j1) reacting the compound of Formula 12 with brominating agent in the presence of a zinc salt catalyst to yield a compound of Formula 4.

6. A process for synthesizing a compound of Formula I 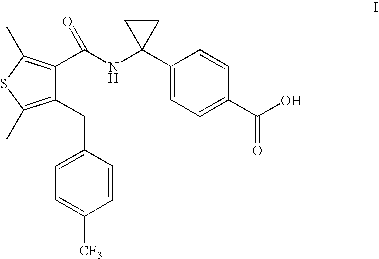
or a pharmaceutically acceptable salt thereof, comprising:
(a2) reacting a compound of Formula 12 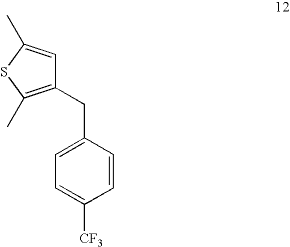
with a compound of Formula 13 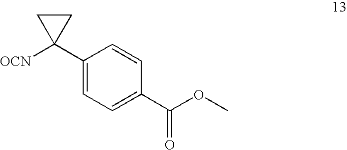
in the presence of a first transition metal salt reagent to yield a compound of Formula 8 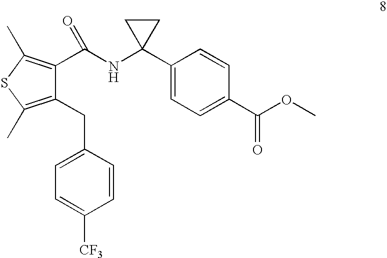
(b2) hydrolyzing the compound of Formula 8 with a strong base of formula X¹—OH or X²—(OH)₂, wherein X¹ is selected from the group consisting of: potassium, cesium, lithium, sodium and rubidium, and X² is selected from the group consisting of: barium, strontium and calcium, followed by acidification to yield the compound of Formula I; and
(c2) optionally reacting the compound of Formula I with a base to yield a pharmaceutically acceptable salt of the compound of Formula I.

7. The process according to claim 6 further comprising making compound of Formula 12 by
(d2) reacting 2,5-dimethylthiophene with a compound of Formula 11 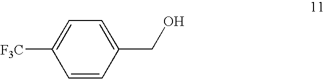
in the presence of a second transition metal salt reagent and a strong acid to yield a compound of Formula 12.

8. The process according to claim 6 further comprising making the compound of Formula 13 by
(e2) reacting a compound of Formula 7 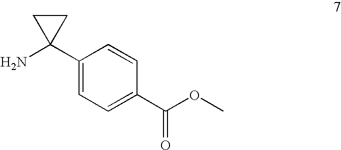
with COCl₂ in the presence of an amine base to yield the compound of Formula 13.

9. The diethylamine salt of the compound of Formula I [chemical structure]
